(19) United States
(12) Patent Application Publication
(10) Pub. No.: US 2026/0315033 A1
(43) Pub. Date: Oct. 15, 2026

(54) Device and method for 3D ultrasound imaging with an array of reprogrammable transducers

(71) Applicants: SA VERMON, Tours (FR); INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); ECOLE SUPÉRIEURE DE PHYSIQUE ET DE CHIMIE INDUSTRIELLES DE LA VILLE DE PARIS, Paris (FR)

(72) Inventors: Benjamin GUERIF, Tours (FR); Clément PAPADACCI, Paris (FR); Thomas DEFFIEUX, Paris (FR); Mickael TANTER, Paris (FR); Mathieu PERNOT, Paris (FR)

(21) Appl. No.: 19/417,789

(22) Filed: Dec. 12, 2025

(30) Foreign Application Priority Data
Dec. 16, 2024 (FR) 2414266

(51) Int. Cl. A61B 8/00; G01S 15/89

(52) U.S. Cl. CPC A61B 8/483; A61B 8/4488; A61B 8/54; G01S 15/8915; G01S 15/8993

(57) ABSTRACT

Three-dimensional ultrasound imaging of a region of interest of a living being is performed with an ultrasound imaging probe having a plurality of elementary ultrasound transducers distributed into a set of groups of elementary transducers. An electronic emission circuit and an electronic reception circuit are connected to the elementary transducers. A (control system communicates with the probe and is configured to program, for each shot: the electronic emission circuit to address a set of elementary transducers for the emission of an unfocused ultrasound wave, and the electronic reception circuit to address all groups of elementary transducers or a distribution of elementary transducers. Sub-apertures capable of making the backscattered waves converge at a focal point located behind the probe, on the side opposite the region of interest to be imaged.

### TECHNICAL FIELD

The present invention relates to a device and method for three-dimensional (3D) ultrasound imaging, in particular to a device and method for 3D ultrasound imaging with an array of reprogrammable transducers enabling ultrafast imaging, with a wide and deep field of view, while at the same time proposing an ergonomic and compact probe.

### PRIOR ART

Ultrasound medical imaging systems have developed very rapidly due to their many benefits for medical diagnostics.

Generally speaking, ultrasound may be generated from a probe comprising an array of a plurality of transducers capable of individually generating ultrasound waves. The ultrasound probe is designed to be held by a practitioner, for example, and moved over various parts of a patient's anatomy in order to obtain the image of the region of interest. The transducers are connected to a control system or ultrasound system by a cable. The ultrasound waves may be directed to a medium, which may generate backscattered signals in response, which may in turn be recorded by the same array of transducers or a different array. The backscattered signals are received and processed by a control system that makes it possible to reconstruct the received signals in the form of a 2D or 3D image. These 2D or 3D images may be displayed in real time on a screen of the control system, thus allowing a practitioner to make a diagnosis.

In conventional three-dimensional imaging, similar to two-dimensional ultrasound imaging, the 3D image may be generated by scanning the volumetric region, successively focusing a plurality of ultrasound beams and then reconstructing each of the corresponding lines onto a volume. At emission, the individual transducers in the array are activated with delays so as to form one or more ultrasound beams. At reception, the signals received by the transducers are delayed and summed in a beamforming phase in order to reconstruct a line of the image. Each transducer is connected to a channel of a beamformer. Each of the formers applies delays to the signals coming from the corresponding transducer so as to orient and focus the beam formed by the former. The signals delayed by each former are combined to form an oriented and focused beam. The multiple beams produced by beamformers operating in parallel are used to form multiple lines of an ultrasound image. In other words, each transducer is associated with a channel on which the received signals are transmitted via a cable to the control system. The signals may be conditioned in the summing electronics module. This approach of associating one summing electronics module per transducer becomes complex and is not feasible in the case of 3D ultrasound imaging over a large volume. Indeed, the ability to image a large 3D volume is related directly to the size of the array of transducers. In order not to lose spatial resolution, it is necessary to use an array of transducers in which the pitch between two transducer elements must be less than the wavelength of the ultrasound wave in order to reduce side lobes and grating lobes, which may impact imaging quality. In addition, imaging a large volume, that is to say with a larger field of view and depth, involves a large aperture. This requirement leads to ultrasound probe configurations comprising thousands of transducers, requiring the use of thousands of channels and a more complex imaging system that would require the use of a large cable to transport thousands of channels. Managing several thousand elements driven by thousands of electronic channels causes problems in terms of cost, wiring complexity and the imaging system used, making this approach difficult to envisage for a clinical application.

In conventional imaging, which is based on focusing ultrasound waves by applying delay and summing operations to ultrasound transducers in order to obtain the desired focusing at emission and reception, one known solution consists in reducing the number of transmission channels between the probe and the control system by integrating, directly into the 2D matrix array, an application-specific integrated processing circuit (ASIC) that makes it possible to perform a first beamforming step at the probe by applying delay and summing operations for groups of transducers, which are also called reception sub-apertures, making it possible to obtain one electrical signal per group of transducers instead of one electrical signal per transducer. In a second step, the electrical signals generated by the micro-beamforming circuits are transmitted to the control system in order to reconstruct a line of the 3D image by again applying delay and summing operations. In other words, the reconstruction of a line of the 3D image is broken down into two steps, a first step at the probe and a second step at the control system. This solution makes it possible to drive an array of several thousand transducers in order to image a large volume with a reduced number of channels suitable for working with a single imaging system. However, this solution is not suitable for ultrafast imaging techniques with a very high imaging rate of the order of 1000 images per second, which are developed to observe very fast phenomena, such as echocardiography, elastography and ultrafast localization microscopy. Indeed, in ultrafast imaging, the reconstruction of the 3D image uses all the signals backscattered by the medium and received by the elements for each shot to reconstruct the entire volume of the region of interest, which is no longer possible after the micro-beamforming step.

Another solution consists in using sparse arrays to reduce the number of transmission and reception channels. This solution consists in addressing some of the elements, from among several thousand transmission and reception elements, so that the number of electrical signals to be processed is compatible with the number of sampling channels of a conventional imaging system. Although such a probe allows ultrafast 3D imaging, using fewer transducers than a so-called fully populated probe gives rise to side lobes and significantly reduces 3D imaging quality. In addition, this solution leads to a reduction in the sensitive surface used at transmission and reception because not all elements are used, resulting in a decrease in signal-to-noise ratio and therefore the quality of the image. For example, this sparse array technique is not suitable for imaging through bones, where there is a need for a maximum amount of transmitted energy and reception sensitivity.

Consequently, capturing large 3D volumes at high imaging rates remains a challenge in ultrasound localization microscopy (ULM), and more particularly in the field of transcranial imaging.

The present invention aims in particular to overcome these drawbacks and to propose an imaging device and method that enable 3D ultrasound imaging with a wide field of view, while at the same time maintaining imaging quality, and suitable for high imaging rates, greater than 1000 images per second, without significantly increasing the complexity and cost of the acoustic imaging devices on which it is implemented.

### SUMMARY OF THE INVENTION

The embodiments described herein make it possible to improve 3D ultrasound imaging devices and methods.

To this end, according to a first aspect, the present disclosure relates to a device for three-dimensional ultrasound imaging of a region of interest of the body of a living being, said device comprising:
an ultrasound imaging probe comprising a plurality of elementary transducers arranged in a matrix in rows and columns forming an array, said elementary transducers being distributed into a set of groups of elementary transducers, an electronic emission circuit and an electronic reception circuit connected to the elementary transducers;
a control system configured to communicate with the probe;
said electronic reception circuit comprising a micro-beamforming circuit for each group of elementary transducers, said micro-beamforming circuit having an input individually connected to each elementary transducer of the group and an output connected to the control system, said micro-beamforming circuit being designed to supply a single signal generated from the response signals of the elementary transducers of the group;
said control system being configured to program, for each shot:
   (a) the electronic emission circuit in such a way as to address a set of adjacent elementary transducers from among the plurality of elementary transducers in order to form an emission aperture and to apply a delay law to the elementary transducers of said set for the emission of an unfocused ultrasound wave;
   (b) each electronic micro-beamforming circuit in such a way as to address the associated group of elementary transducers or an elementary transducer of the group in order to form a reception sub-aperture having a focal point located behind the array, on the side opposite the region of interest to be imaged, designed to receive backscattered waves, such that a plurality of reception sub-apertures are formed.

In the present invention, the array of elementary transducers is divided into a set of groups, each group being associated with a dedicated micro-beamforming circuit. At reception, each micro-beamforming circuit forms a reception sub-aperture from the transducers of the associated group, and all of these sub-apertures work in parallel. The number of reception sub-apertures formed is equal to the number of groups of transducers. Thus, applying the delay to the elementary transducers within each group makes it possible to obtain a sparse array of reception sub-apertures each having a focal point behind the array, on the side opposite the region of interest to be imaged. The simultaneous formation of multiple reception sub-apertures, each focused virtually behind the array, makes it possible to increase the reception area, to reduce grating lobes and to improve the quality of the 3D image, while at the same time limiting electronic complexity and the number of transmission channels. This architecture, in which each group of transducers forms a distinct reception sub-aperture, results in a total number of sub-apertures corresponding to the number of groups. In other words, at each shot, at reception, all the groups of transducers of the probe are addressed by the control system to form a plurality of unfocused reception sub-apertures.

Moreover, the control system of the invention is configured to reprogram, at each shot, the emission aperture and the configuration of the reception sub-apertures, which is different from the configuration of the emission aperture. This flexibility makes it possible to dynamically adapt geometry and focusing according to imaging needs.

The features set forth in the following paragraphs may optionally be implemented independently of one another or in combination with one another:

The control system may be configured to reprogram the electronic emission circuit to modify the emission aperture and the emission delays to emit an unfocused ultrasound wave at each shot.

The control system may be configured to reprogram the electronic micro-beamforming circuits to modify the reception sub-apertures and the reception delays to receive backscattered waves at each shot.

The control system may be configured to program the electronic emission circuit to apply emission delays to the transducers so that the emitted unfocused ultrasound wave is a plane wave or divergent wave.

Each micro-beamforming circuit may comprise a reception and delay circuit for each transducer of the group and a signal micro-summing circuit.

Preferably, the reception and delay circuits of the group are capable of applying a delay law to the elementary transducers of the group in such a way as to form a virtual lens the focal point of which is located behind the array, on the side opposite the region to be imaged.

Thus, at reception, a plurality of virtual lenses are formed. The number of virtual lenses is equal to the number of groups of transducers of the array.

Moreover, the control system may be configured to program the reception and delay circuits such that the virtual lenses that are formed have identical focal lengths.

According to one variant, the control system may be configured to program the reception and delay circuits such that at least two virtual lenses have different focal lengths.

According to yet another variant, the control system is configured to program the reception and delay circuits such that at least two virtual lenses have different focusing directions.

According to the present disclosure, the control system may be configured to program the receiving and delay circuits so that a plurality of virtual lenses have focus points randomly distributed behind the transducer array, on the side opposite to the region of interest to be imaged. Thus, advantageously, these combinations of random arrangements of the focus points of the virtual lenses disrupt the periodicity of the system, which makes it possible to improve image quality in 3D volumetric ultrasound imaging.

According to one embodiment, the reception and delay circuit comprises a reception amplifier, a time gain compensation circuit and a delay circuit.

According to another aspect, the disclosure relates to a method for three-dimensional (3D) ultrasound imaging of a region of interest of the body of a living being using a three-dimensional ultrasound imaging device as defined above, said method comprising, for each shot:
(a) a programming step during which the electronic emission circuit is programmed (PROGRAM_EMI) to address a set of adjacent elementary transducers from among the plurality of elementary transducers in order to form an emission aperture and to apply a suitable delay law to the elementary transducers of said set for the emission of an unfocused ultrasound wave;
(b) a programming step during which each electronic micro-beamforming circuit is programmed to address the associated group of elementary transducers or at least one elementary transducer of the group in order to form a reception sub-aperture having a focal point located behind the array, on the side opposite the region of interest to be imaged, designed to receive backscattered waves (PROGRAM_REC), such that a plurality of reception sub-apertures are formed;
(c) an emission step during which the unfocused ultrasound wave is emitted in the direction of the region of interest through said emission aperture formed by said set of addressed adjacent elementary transducers (EMI_US);
(d) a reception step during which the backscattered waves coming from the region of interest are received by each reception sub-aperture formed in the probe (REC_US);
(e) a signal transmission step during which the signal supplied by each micro-beamforming circuit is transmitted to the control system (TRANS_SIG);
(f) a 3D image construction step during which the signals are delayed and summed in the control system (140) in order to construct a 3D ultrasound volumetric image corresponding to the emitted wave (CONST_IMG_COM).

Moreover, the method may comprise any of the following features:

Steps (a) to (f) may be repeated in order to emit a series of unfocused ultrasound waves in various propagation directions and to acquire a series of signals for each emitted wave, said signals making it possible to form a series of 3D volumetric images, and wherein the method furthermore comprises a step of constructing a composite image during which said 3D volumetric images are summed coherently in order to form a final composite image.

The control system may reprogram the emission circuit to modify the emission aperture and the emission delays to emit an unfocused ultrasound wave at each shot.

The control system may reprogram the electronic micro-beamforming circuit to modify the reception sub-apertures and the reception delays to receive backscattered waves at each shot.

### BRIEF DESCRIPTION OF THE DRAWINGS

Other features, details and advantages will become apparent on reading the detailed description below and on analysing the appended drawings, in which:

FIG. 1 shows a block diagram of a 3D ultrasound imaging device according to one embodiment;

FIG. 2 shows the micro-beamforming circuit of the device of FIG. 1 in more detail;

FIG. 3 shows the elementary reception and delay circuit of FIG. 2 in more detail;

FIG. 4A shows a schematic plan view of the transducers of the probe of FIG. 1 in an emission step in which all the transducers are used to emit an ultrasound wave in such a way as to form an emission aperture;

FIG. 4B shows a schematic plan view of an array of transducers of the probe of FIG. 1 in an emission step in which some of the peripheral transducers are not used in such a way as to form a dense emission aperture, but with a geometric shape different from that of the emission aperture of FIG. 4A;

FIG. 5A shows a schematic side view of the transducers of the probe of FIG. 4A in a step of emitting a plane wave;

FIG. 5B shows a schematic side view of the transducers of the probe of FIG. 4A in a step of emitting a divergent wave;

FIG. 5C shows a schematic side view of the transducers of the probe of FIG. 4B in a step of emitting a divergent wave;

FIG. 6A shows a schematic view of the elementary transducers of the probe of FIG. 2 in a reception step in which the micro-beamforming circuit is programmed by the control system such that each subset of elementary transducers emulates a virtual acoustic lens so as to make an acoustic wave in the reception phase converge at a virtual focal point located at the rear of the array, on the side opposite the external medium to be imaged, the radius of curvature and orientation of all the convergent virtual lenses being identical;

FIG. 6B shows a schematic view of the elementary transducers of the probe of FIG. 2 in a reception step in which the micro-beamforming circuit is programmed by the control system such that the subsets of transducers emulate an array of convergent virtual lenses, configured so as to make an acoustic wave in the reception phase converge at a virtual focal point located at the rear of the array, on the side opposite the external medium to be imaged at reception with a radius of curvature greater than that of FIG. 6A, the radius of curvature and orientation of all the convergent virtual lenses being identical;

FIG. 6C shows a schematic view of the transducers of the probe of FIG. 2 in a reception step in which the micro-beamforming circuit is programmed by the control system such that the subsets of transducers form an array of convergent virtual lenses, configured so as to make an acoustic wave in the reception phase converge at a virtual focal point located at the rear of the array, on the side opposite the external medium to be imaged at reception, the radius of curvature and orientation of the virtual lenses varying from one lens to another in a pseudorandom manner predefined in accordance with an algorithm;

FIG. 7A shows a schematic plan view of the 2D array of FIG. 2 in a reception step in which the micro-beamforming circuit of each group is programmed by the control system to address one transducer of each group of elementary transducers, the addressing pattern being identical for all the groups of the array and identical for each shot;

FIG. 7B shows a schematic plan view of the 2D array of FIG. 2 in a reception step in which the micro-beamforming circuit of each group is programmed by the control system to address one transducer of each group of elementary transducers, the addressing pattern being different from one group to another and different for each shot;

FIG. 8 shows the main steps of an ultrafast imaging method implementing the device of FIG. 3 in the case where the groups of elementary transducers are controlled individually so as to form unfocused reception sub-apertures or emulate divergent virtual lenses.

### DESCRIPTION OF THE EMBODIMENTS

### Definitions

In the figures, the same references designate identical or similar elements.

In the present disclosure, the term “aperture” as used herein designates an available aperture formed by a set of transducers arranged in a two-dimensional array, the aperture being used to emit ultrasound waves and/or receive backscattered signals.

In the present disclosure, the term “sub-aperture” as used herein designates an aperture formed by a group of transducers, an emission sub-aperture being formed by a subset of elementary transducers of the two-dimensional array in the phase of emitting an ultrasound wave, a reception sub-aperture being formed by a subset of elementary transducers in the phase of receiving signals backscattered by the medium.

In the present disclosure, the term “dense sub-aperture” as used herein designates an aperture formed by a group of adjacent transducers.

In the present disclosure, “micro-beamforming” is understood to mean a delay and sum beamforming strategy broken down into two steps. The first step is carried out in the ultrasound probe and is carried out in analogue or digital form by a set of dedicated electronic micro-beamforming circuits, each for forming an unfocused sub-aperture. The signals thus micro-formed, that is to say delayed and summed in the probe, are transmitted from the probe to a control system external to the probe. The second step is carried out in the control system by again applying a beamforming strategy based on applying delays and sums between the micro-formed signals to reconstruct a 3D image.

In the present disclosure, the term “unfocused reception sub-aperture” as used herein designates a group of elementary transducers the generated response signals of which are delayed and summed in such a way so as to emulate a virtual lens the virtual focal point of which is located behind the array of elementary transducers, on the side opposite the region to be imaged.

In the present disclosure, the term “virtual lens” as used herein designates a virtual acoustic lens having a curved shape oriented towards the external medium to be imaged and capable of making a beam of ultrasound waves converge, in the reception phase, at a focal point located behind the array of transducer elements, on the side opposite the region to be imaged.

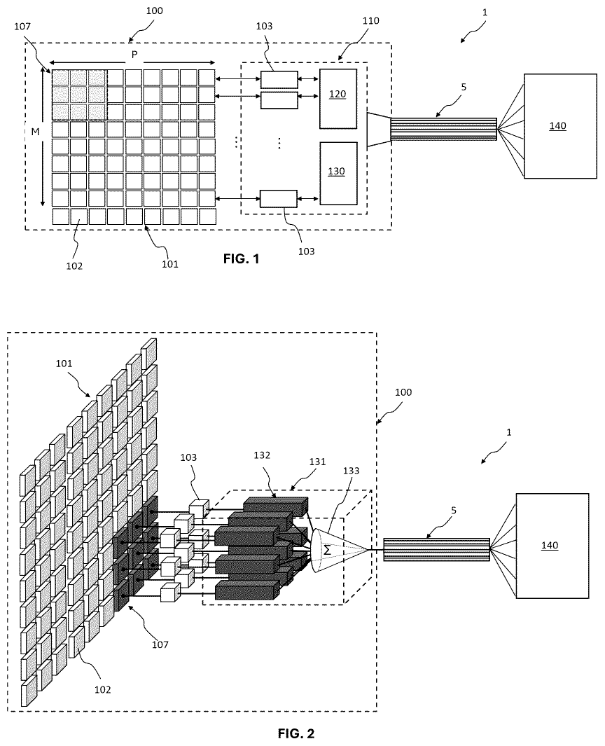

FIG. 1 is a schematic view showing one example of an ultrasound imaging device 1 configured to produce a three-dimensional (3D) ultrasound image of a medium to be imaged by emitting and receiving ultrasound waves.

The medium to be imaged designates for example a region of interest of the body of a patient or animal. By way of example, the device presented is designed to carry out ultrafast imaging using 3D or 4D (3D+time) three-dimensional ultrasound on the heart of a living being.

The imaging device 1 may comprise a probe 100 and a control system 140 connected to the probe via a cable 5.

The probe 100 comprises a two-dimensional (2D) array 101 of elementary ultrasound transducers 102 and an electronic control circuit for controlling the elementary ultrasound transducers 110. The electronic control circuit 110 comprises an electronic emission circuit 120 designed to apply an electrical excitation signal to the elementary transducers in order to emit an ultrasound wave, an electronic reception circuit 130 designed to control the elementary transducers in order to receive backscattered waves, and a plurality of emission/reception switches 103 for connecting the elementary ultrasound transducers 102 either to the emission circuit 120 or to the reception circuit 130.

The control system 140 is configured to control the electronic emission and reception circuits 120, 130 in such a way as to program the activation or extinction of the elementary ultrasound transducers of the ultrasound probe 100 in order to modify the emission aperture and the reception aperture before each shot, and to record and process the backscattered signals transmitted by the probe in order to generate a three-dimensional image of the medium of interest.

The control system 140 may for example comprise a control unit and a computer. The control unit is used to control the electronic emission and reception circuits 120 and 130 and acquire and process the signals transmitted by the probe 100, whereas the computer is used to control the control unit and construct 3D images from the signals acquired and processed by the control unit and determine quantization parameters from the 3D images. According to one variant and as illustrated in FIG. 1, a single electronic device 140 may fulfil all the functionalities of the control unit and of the computer. According to another variant, at least some of the functionalities of the control unit and of the computer may be integrated into the probe 100.

The cable 5 makes it possible to connect the probe 100 to the control system 140 in order to transmit control instructions to the electronic emission circuit 120 and the electronic reception circuit 130 housed in the probe in order to control the elementary transducers individually. According to one exemplary embodiment, the cable 5 also makes it possible to transmit the electrical signals acquired by the probe 100 from the probe 100 to the control system 140. According to one variant, a wireless link may replace the cable 5.

The imaging device may furthermore comprise a screen or any other user interface for visualizing an image of the field of view.

The probe 100 is for example a portable probe that may be positioned and/or moved by the practitioner over the body of the patient or animal in order to image a region of interest.

The probe 100 may comprise a plurality of elementary ultrasound transducers 102 arranged in a matrix in M rows and P columns in the form of an array 101. The elementary transducers are for example all substantially identical. The pitch between two transducers is of the order of the wavelength in the direction of the rows and in the direction of the columns. One example of a matrix 101 of 9*9 elementary transducers is shown in FIG. 1. In practice, the matrix may comprise several thousand to several tens of thousands of transducers, which may be addressed individually by the electronic emission circuit 120 and the reception circuit 130 in order to emit an ultrasound wave and receive an ultrasound wave backscattered by the insonified medium. The probe may comprise for example 1024 transducer elements (32*32), with a pitch of 0.3 mm. The transducers may emit for example at a centre frequency between 1 and 10 MHz, for example 3 MHz.

In the example of FIG. 1, the elementary transducers have a square shape and the inter-transducer pitch is identical in the direction of the rows and in the direction of the columns. According to another exemplary embodiment, the inter-transducer pitch may be different in the two directions, and the transducers may have a rectangular shape. The following description will be given using the example of FIG. 1, but other forms of transducer arrays are also possible within the scope of the present disclosure.

As is known, each elementary transducer comprises two electrodes for applying an electrical excitation signal to the transducer and for reading an electrical response signal from the transducer. The emission/reception switch 103 is configured to connect an electrode of the transducer 102 of the matrix 101 either to an output terminal of the electronic emission circuit 120 during the phase of emitting an ultrasound wave, or to an input terminal of the reception circuit 130 during the phase of receiving an ultrasound wave.

During operation, the set of transducers 102 is placed on a region of interest of a patient the 3D image of whom it is desired to acquire. The electronic emission circuit 120 is configured to apply pulses to the transducers so as to cause ultrasound waves to be emitted in the direction of the region of interest. The ultrasound waves emitted by the transducers are backscattered by the insonified medium and received by the transducers, which convert them into electrical signals, referred to as raw RF signals. The emission step and the reception step form a shot or a measurement step. The raw RF signals are received by the electronic reception circuit 130 at the probe in order to be preformed and then transmitted to the control and processing device 140 via the cable 5 for the purpose of reconstructing a 3D image from said preformed signals.

In the present disclosure, in order to be able to implement ultrafast imaging, the imaging system 140 of the present disclosure is configured to program the electronic emission circuit 120 such that, in a emission step, the elementary transducers of the matrix are activated so as to form an emission aperture suitable for the emission of an unfocused wave, that is to say a plane wave or divergent wave to insonify (acoustically excite) the whole medium. The signal picked up by each transducer comes from the entire medium. Moreover, in order to keep the probe compact and limit the number of reception channels, the control system 140 is configured to program the electronic reception circuit 130 such that, in a reception step, the elementary transducers of the matrix are activated so as to form reception sub-apertures suitable for transmitting a number of micro-formed response signals compatible with the number of sampling channels available on the control system 140. The control system 140 is configured to generate a volume or a 3D image corresponding to the transmitted wave from the acquired micro-formed response signals by applying a delay and summing law.

In order to increase imaging quality, the control system 140 is configured to program the electronic emission circuit 120 and the electronic reception circuit 130 in such a way as to repeat the emission step and the reception step at a high rate, so as to make the transducer array successively emit a series of N unfocused plane or divergent ultrasound waves respectively having different propagation directions so as to perform N shots in order to obtain N acquisitions of electrical signals. The N acquisitions of electrical signals make it possible to form N 3D images or N volumes, which are then summed coherently to form a high-quality composite image.

Advantageously, the control system 140 is configured to reprogram the electronic emission circuit 120 and the electronic reception circuit 130 between two measurement steps or two shots in such a way as to respectively modify the emission aperture and the reception sub-apertures formed by groups of transducers or the reception aperture formed by a sparse distribution of distributed transducers from among all the transducers of the array in order to increase imaging sensitivity.

The control system 140 is configured to then coherently sum the N angled 3D images or the N volumes obtained after each shot in order to generate a high-quality final 3D image of the region.

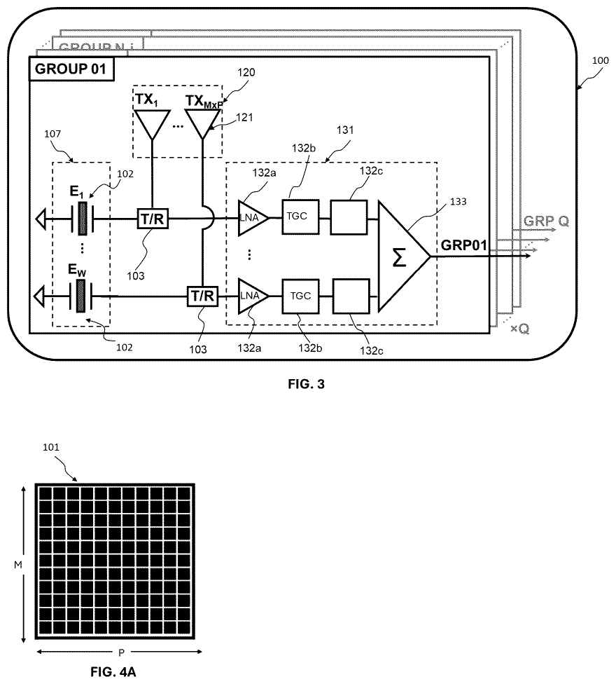

With reference to FIG. 3, the emission circuit 120 comprises an elementary emission circuit 121 for each transducer.

In the emission phase, the emission/reception switch 103 connects an electrode of the elementary transducer to an output terminal of the elementary emission circuit. An electrical excitation signal is applied to the elementary transducer by the elementary emission circuit. Under the effect of the electronic pulse, each of the elementary transducers generates a spherical acoustic wave that propagates in front of it and that is added to the contribution of those of its neighbours. The control system 140 is configured to independently control each elementary transducer via the elementary emission circuit so as to add a particular delay such that the spherical waves emitted by each of the elementary transducers are superimposed so as to form a particular wavefront, for example an unfocused ultrasound wave, for example a plane wave or a divergent wave, in order to insonify the entire medium.

According to another embodiment, the control system 140 is configured to program the elementary emission circuits 121 in such a way as to independently drive each elementary transducer so as to choose the transducers to be activated in order to obtain a particular dense emission aperture with a particular geometry. This activation may be achieved for example by increasing or decreasing the amplitude of the excitation signal transmitted to the corresponding transducer, or even by imposing a zero amplitude, this corresponding to the extinction of the channel associated with the elementary transducer. A dense emission aperture designates the configuration in which the aperture is obtained using a group of adjacent transducers of the matrix of the probe. According to another embodiment, the control and processing device 140 is configured to program the elementary emission circuits 121 such that all the transducers of the matrix are used to form the emission aperture.

Advantageously, the control system 140 is also configured to program the elementary emission circuits 121 in such a way so to apply emission delays to the transducers so as to adjust the radius of curvature of the divergent wave and/or the propagation direction of the plane wave or divergent wave.

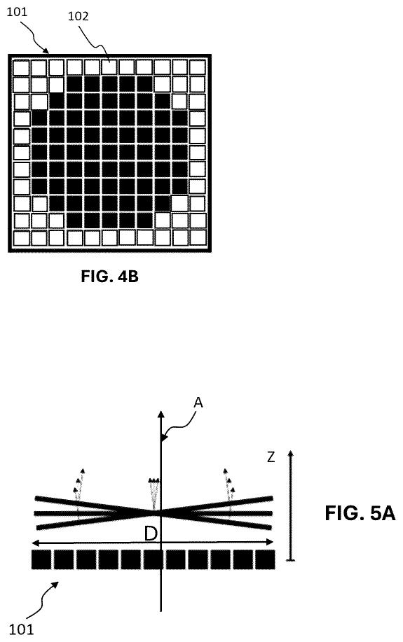

FIG. 4A shows one example in which the emission aperture is obtained by using or addressing all the transducers of the array in an emission state, making it possible to obtain a higher power of the ultrasound wave. In FIG. 4A, the dark squares represent activated transducers or transducers used to emit a plane wave or spherical wave.

FIG. 4B shows one example in which the transducers located at the periphery of the array are not used, thus making it possible to form a dense emission aperture with a geometry different from that of FIG. 4A. In FIG. 4B, the white squares represent the non-activated elementary transducers and the dark squares represent the elementary transducers that are activated so as to emit a plane wave or spherical wave, and which therefore contribute to the formation of the unfocused wave. The unused transducers may correspond to transducers the associated channels of which are switched off or to transducers for which the amplitude of the transmitted excitation signal is weighted. Particularly advantageously, the amplitude of the signal is reduced for the transducers at the periphery of the dense emission aperture in such a way as to reduce the edge effects of the emitted wave.

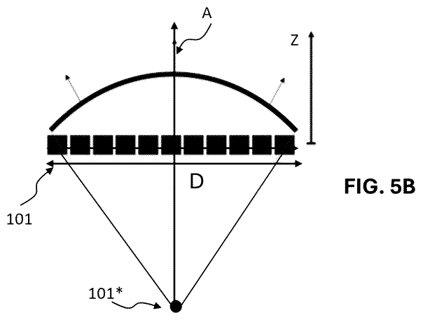

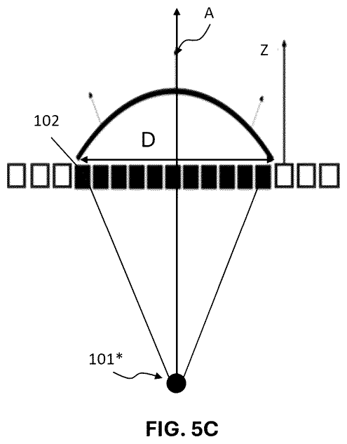

FIGS. 5A-5C illustrate a schematic side view of the array of FIG. 1 with different emission apertures and different emission delays.

According to one embodiment and with reference to FIG. 5A, the elementary emission circuits are programmed by the control system 140 to activate all the elementary transducers of the matrix, namely the 9*9 elementary transducers with a maximum emission aperture (D) and to add an emission delay to the elementary transducers in such a way as to emit a plane ultrasound wave in various propagation directions with respect to the acoustic axis of the array A. When the elementary emission circuits transmit an electronic pulse without a phase shift to the transducers of the array, the plane wave is emitted in the direction of the axis A. The entire medium is therefore insonified in a single shot, and the whole medium backscatters the ultrasound wave.

In order to increase imaging quality, the elementary emission circuits are programmed by the control system 140 such that the plane wave is emitted by the array in multiple directions forming an angle with the acoustic axis A so as to incline the wavefront. The medium is thus insonified with a succession of angled plane waves, and the reconstructed image for each of the plane waves is an angled base image. The elementary emission circuits transmit a phase-shifted electronic pulse to the transducers of the array such that the plane wave is emitted at a different angle with respect to the direction of the axis A. Coherently summing all the angled base images makes it possible to create synthetic focusing and makes it possible to reconstruct a higher-quality composite image with a high imaging rate. FIG. 5A shows a plane wave at 0° and two angled plane waves.

According to another embodiment and with reference to FIG. 5B, the elementary emission circuits are programmed to add a delay to the elementary transducers in such a way as to emit a divergent ultrasound wave having a convex wavefront, making it possible to insonify a field wider than that of the plane wave. The elementary emission circuits are programmed by the control system 140 in such a way as to send a delay to the transducers. The delay law applied to the elementary transducers 102 is inverted with respect to focused imaging. In other words, elementary transducers located in the centre will shoot first, and transducers located at the periphery will shoot last. This is tantamount to considering a virtual source 101* positioned behind the array on its axis A, on the side opposite the medium to be imaged.

As in the case of the plane wave, in order to increase imaging quality, the elementary electronic emission circuits are programmed by the control system 140 such that the divergent wave is emitted along axes that deviate from the axis A of the transducer array. This is tantamount to moving the virtual source from one side of the axis A to the other so as to move the centre of curvature of the divergent wave. The medium is thus insonified with a succession of divergent waves with different orientations, and the reconstructed image for each of the divergent waves is a base image. Coherently summing all the formed base images makes it possible to reconstruct a final composite image of better quality.

According to another embodiment and with reference to FIG. 5C, the elementary emission circuits are programmed by the control system 140 such that the peripheral elementary transducers are not activated as in the case of FIG. 4B and to add a delay to the activated elementary transducers in such a way as to emit a divergent ultrasound wave having a convex wavefront.

For each transmitted signal, backscattered signals may be generated by the medium in response to the transmitted signal. The backscattered signals are received by all the transducers of the array, namely the M*N transducers of the array that generate electrical signals. These signals are received by the reception circuit 130 and may be processed using a beamforming strategy to form an image.

FIG. 2 illustrates the reception circuit 130 according to one embodiment in more detail.

The reception circuit 130 comprises a set of micro-beamforming circuits 131 located directly in the probe 100, which makes it possible to reduce the number of response signals to be transmitted from the probe 100 to the control system 140 by transmitting, to the control system 140, signals coming from a set of unfocused reception sub-apertures formed by groups of transducers or elementary transducers chosen from each group. In other words, in the reception phase, the micro-beamforming circuits 131 create a virtual reception array with a reduced number of signals to be transmitted from the probe 100 to the control system 140.

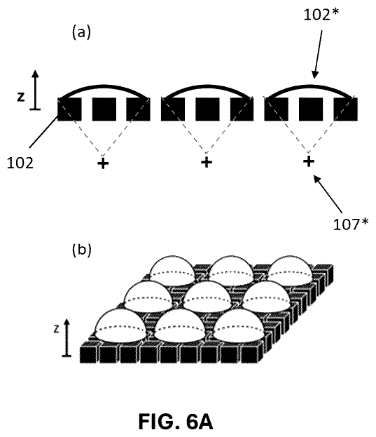

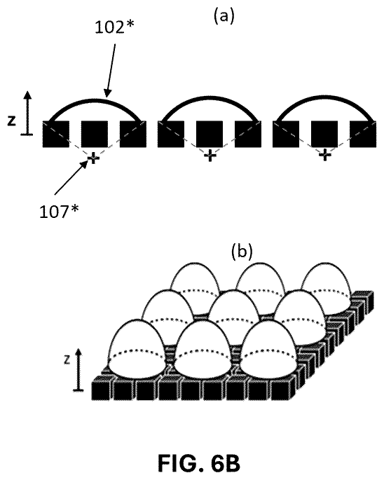

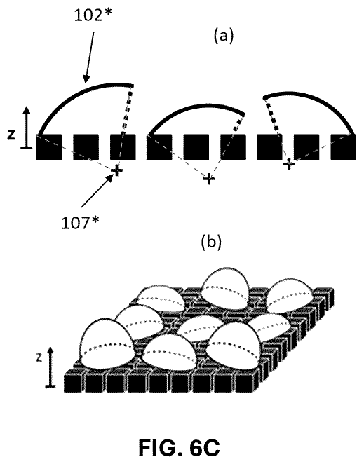

According to one embodiment, the signal coming from the array of virtual reception transducers may correspond to the backscattered signals received by all the transducers forming the reception sub-apertures, as illustrated in FIGS. 6A-6C.

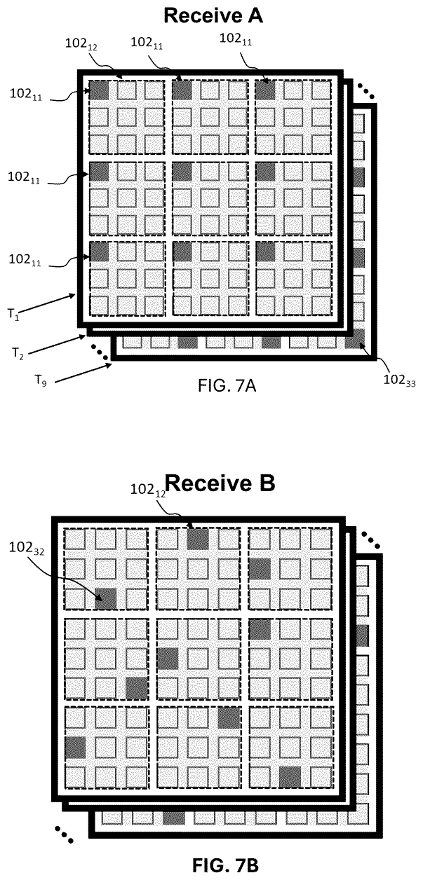

According to another embodiment, the signal coming from the virtual reception array may correspond to the backscattered signals received by one of the elementary transducers of each group, the transducers being chosen according to an ordered pattern or according to a random pattern, as illustrated in FIGS. 7A and 7B.

The micro-beamforming circuit 131 thus makes it possible to break down the process of forming a 3D image into two steps, a first step of partially forming, at the probe, a number of electrical signals from among the electrical signals generated by all the transducers of the array in such a way as to obtain a number of signals to be transmitted from the probe to the control and processing device 140 less than or equal to the number of channels available on the control system 140, and a second step of reconstructing the 3D image in the control system 140 from the micro-formed signals coming from the reception sub-apertures formed by groups of elementary transducers or by elementary transducers chosen from among the elementary transducers of the array. In other words, after a wave shot, the backscattered ultrasound wave is picked up by each transducer of the array. The signal thus picked up by each transducer comes from the entire medium, since the incident wave is a plane wave or divergent wave. The backscattered signals are picked up by the M*P transducers, for example by 9*9 transducers in the case of FIG. 1. The number of M*P signals picked up by the transducers are processed in the probe in such a way as to supply a number Q of signals, Q being less than M*P, and at most equal to the number of channels available on the control system 140. The Q signals formed are transmitted on the signal transmission channels of the cable 5 to the control system 140 for processing purposes in order to reconstruct a 3D image or a volume corresponding to the incident wave. According to one exemplary embodiment, a time division multiplexing circuit may connect the output of the reception circuit 130 to the signal transmission channels of the cable 5. Time division multiplexing (TDM) consists in extracting a temporal portion of N from among the Q signals—N being an integer greater than 2 and less than or equal to Q—and in transmitting the N temporal portions one after another in one and the same channel of the cable 5. This multiplexing advantageously makes it possible to reduce the number of channels making up the cable 5.

By using reception sub-apertures or using a number of elementary transducers chosen from within the limit of available channels, it is possible to use a large number of elementary transducers to carry out 3D volumetric imaging with a large field of view, without increasing the complexity of the drive electronics and the size thereof, making them compatible with clinical use.

Furthermore, in order to increase the reception angle of the waves backscattered by each group of elementary transducers while still maintaining a sensitive surface equivalent to that of all the transducers of this group and the spatial resolution of the 3D image, each group of elementary transducers emulates a virtual acoustic lens or a virtual transducer element having a reception surface of convex curved shape oriented towards the region of interest or medium to be imaged. More specifically, the micro-beamforming circuit 131 is programmed by the control system 140 such that the elementary transducers of the group are controlled independently in order to emulate a virtual transducer having a reception surface of convex curved shape or a virtual lens capable of making the received wave converge at a focal point located behind the array, on the side opposite the medium to be imaged.

With reference to FIG. 2, a micro-beamforming circuit 131 is described below according to one embodiment.

According to one exemplary embodiment, the elementary transducers of the array are distributed into groups 107, thus each forming a sub-matrix of n*n adjacent transducers. In the example of FIG. 2, the sub-matrix is a square and n is equal to 3. Each elementary transducer 102 belongs to one and just one elementary group 107. The reception circuit 130 therefore comprises an electronic micro-beamforming circuit 131 for each group of elementary transducers. In the example of FIG. 2, the reception circuit 130 therefore comprises nine electronic micro-beamforming circuits 131 each associated with a group of elementary transducers.

The micro-beamforming circuit 131 comprises an elementary reception and delay circuit 132 for each elementary transducer of the group 107, that is to say n*n elementary delay circuits, and one elementary micro-summing circuit 133 per group. As illustrated in FIG. 2, this micro-beamforming circuit 131 is individually connected to each elementary transducer of the group via an emission/reception switch 103 and configured to supply an analogue or digital signal corresponding to a sum of response signals of the elementary transducers of the group.

Each elementary reception and delay circuit 132 has an input terminal connected to the emission/reception switch 103 and an output terminal. Each elementary reception and delay circuit 132 is designed to supply, on its output terminal, a conditioned response signal delayed by a predefined reception delay.

Each micro-summing circuit 133 comprises one input terminal per elementary transducer of the associated group, that is to say n*n input terminals, and a single output terminal. The input terminals of the summing circuit are connected to the output terminals of the elementary reception and delay circuits 132.

The summing circuit 133 is designed to supply, on its output terminal, a signal corresponding to the sum of the n*n conditioned and delayed response signals applied to its input terminals.

In the example of FIG. 2, each group comprises a sub-matrix of 3*3 elementary transducers. Each micro-beamforming circuit 131 therefore comprises 9 elementary reception and delay circuits 132.

FIG. 3 illustrates the elementary reception and delay circuit 132 of FIG. 2 according to one embodiment in more detail. As is known, the electronic micro-beamforming circuit may be an application-specific integrated circuit (ASIC).

In the example of FIG. 3, the elementary transducers of the array are distributed into Q groups of elementary transducers. Each group comprises W elementary transducers.

The reception and delay circuit 132 may comprise a reception amplifier 132a, a time gain compensation (TGC) circuit 132b and a delay circuit 132c.

The reception amplifier 132a may be for example a low-noise linear amplifier.

The time gain compensation circuit 132b is configured to apply a variable analogue gain as a function of time to the response signal of the elementary transducer during the reception step.

Each delay circuit 132c has an input terminal connected to the output terminal of the time gain compensation circuit and an output terminal connected to the summing circuit 133. The delay circuit 132c is designed to supply, on its output terminal, a signal corresponding to the signal applied to its input terminal delayed by a predetermined duration. The delay circuits 132c within each group 107 are programmed by the control system 140 to apply a delay law to the elementary transducers of the group such that the group emulates a virtual lens capable of making the received wave converge at a focal point located behind the array, on the side opposite the medium to be imaged. The reception amplifier circuits 132a and the time gain compensation circuits 132b of each group 107 are also programmed by the control system 140 so as to balance the amplitude of the signals of each elementary transducer of the group, or even to deactivate the contribution of one or more elementary transducers of the group, as will be detailed below.

The micro-beamforming circuits 131 are thus configured to form a set of unfocused reception sub-apertures or a set of virtual lenses. In other words, the control system 140 receives response signals transmitted by a new virtual array that makes it possible to reduce the number of channels while still maintaining sensitivity by providing unfocused reception apertures.

By way of example, the delay circuit 132c may be based on the use of switched capacitors. In this context, the use of convergent analogue delays advantageously makes it possible to reduce the maximum analogue delays required per sub-aperture, and consequently the complexity of the ASIC of each associated delay circuit 132c. Indeed, analogue delays are related to the number of capacitors that take up space in the ASIC. Limiting delays makes it possible to increase the compactness of the ASIC or to add new functionalities to the probe.

According to one embodiment, the micro-beamforming circuit 131 may furthermore comprise an analogue-to-digital converter (not shown) at the output of the micro-beamforming circuit 131. In this case, the signals supplied by the micro-beamforming circuits 131 are transmitted to the control system 140 in digital form.

In the example of FIG. 3, the micro-beamforming circuit 131 therefore comprises, for each group, 9*9 reception amplifiers 132a, 9*9 time gain compensation circuits 132b, 9*9 delay circuits 132c, and a summing circuit 133.

FIGS. 6A to 6C schematically illustrate three examples of arrays of virtual lenses 102* having a virtual reception surface of convex curved shape oriented towards the medium to be imaged and having a focal point placed at the rear of the matrix, on the side opposite the region to be imaged. Such virtual lenses may make it possible to reduce directivity by acting as a virtual lens. The virtual focal point 107* located behind the array may be adjusted by choosing the radius of curvature of the curved shape.

FIG. 6A shows a side view (a) of the matrix of FIG. 2 and a perspective view (b) of this matrix. In the example of FIG. 6A, applying the delay to the elementary transducers within each group 107 makes it possible to obtain a sparse array of nine virtual lenses 102*. Such micro-beamforming makes it possible to maintain sensitivity by using all the transducers at reception while at the same time reducing the number of channels. In the example of FIG. 6A, the virtual lenses 102* that are obtained have a convex curved surface having identical radii of curvature and identical orientations.

FIG. 6B illustrates another example of the formation of virtual lenses 102* having a convex reception surface with a radius of curvature greater than that of the virtual lenses of FIG. 6A. As in the example of FIG. 6A, the virtual lenses 102* that are obtained have a convex curved surface having the same radii of curvature and the same orientations.

FIG. 6C illustrates another example of the formation of virtual lenses 102* having a random distribution of radii of curvature and orientations.

The exemplary embodiments shown in FIGS. 6A to 6C make it possible to collect the backscattered signal in a wider angular sector while still maintaining sensitivity. The improvement in directivity of the sub-apertures at reception makes it possible to reduce the amplitude of grating lobes, and therefore to improve image quality. The dynamic variation of the curvature of the virtual lenses in the case of FIG. 6A and FIG. 6B makes it possible to optimize the image reconstruction as a function of depth. The pseudorandom variation of the distribution of virtual sources resulting in an array of virtual lenses with different curvatures and orientations in FIG. 6C also makes it possible to reduce the periodicity of the array at reception and therefore the amplitudes of the associated grating lobes.

The signals, delayed appropriately by each delay circuit 132c, are then summed in analogue form by the micro-summing circuit 133 so as to form a micro-formed signal.

The micro-formed signal supplied by each micro-beamforming circuit 131 is then transmitted to the control system 140 via the cable 5. Micro-beamforming thus makes it possible to reduce the number of response signals to be transmitted while still maintaining the sensitivity of the probe. Each group of transducers supplies a micro-formed signal representative of the sum or superposition of the analogue signals applied to the input terminals of the micro-summing circuit.

The control system 140 may then reconstruct a volume or a 3D image by applying a delay and summing operation to the micro-formed signals coming from the groups of transducers. In other words, the control system applies macro-beamforming to signals coming from a virtual array made up of virtual lenses, which are macro-elements each corresponding to a group of elementary transducers. The control system 140 may comprise, as in the probe, a macro-beamforming circuit comprising a reception and delay circuit for each group and a summing circuit. The reception and delay circuit may comprise for example a reception amplifier, preferably a low-noise amplifier (LNA), a time gain compensation (TGC) circuit and a delay circuit.

To improve the resolution and contrast of the image, it is useful to transmit the unfocused ultrasound waves in series of successive unfocused ultrasound waves, the successive unfocused ultrasound waves of each series having different propagation directions, respectively. In this case, each 3D image is synthesized from the signals acquired from the series.

The successive ultrasound waves of each series may be obtained by varying the virtual source 101* from one wave to another, varying the wavefront as shown in FIGS. 5A-5C. Each series may comprise for example 1 to 81 successive ultrasound waves of different directions, for example 3 to 25 successive ultrasound waves of different directions, for example 5 to 20 successive ultrasound waves of different directions, for example 10 to 20 successive ultrasound waves of different directions.

After each emission of ultrasound waves, backscattered signals are acquired by the 2D array. These raw data are used to generate a sequence of images.

After having received the backscattered signals, beamforming may be applied by the control system 140 to reconstruct the 3D image for each ultrasound wave emitted. The 3D images are then composed coherently so as to form a high-quality final 3D image. The principle of this synthetic imaging is known and is described for example in:
Papadacci, C., Pernot, M., Couade, M., Fink, M. & Tanter, M. High-contrast ultrafast imaging of the heart. IEEE transactions on ultrasonics, ferroelectrics, and frequency control 61, 288-301, doi:10.1109/tuffc.2014.6722614 (2014).
Montaldo, G., Tanter, M., Bercoff, J., Benech, N., Fink, M., 2009. Coherent plane-wave compounding for very high frame rate ultrasonography and transient elastography. IEEE Trans. Ultrason. Ferroelectr. Freq. Control 56, 489-506. doi:10.1109/tuffc.2009.1067.

According to one embodiment, the control system 140 is configured to program the electronic emission circuit 120 and the electronic reception circuit 130 in such a way as to repeat the emission step in order to emit a series of N unfocused, plane or divergent ultrasound waves having different propagation directions, and the reception step in order to receive N raw signals in order to construct a series of N 3D images or N volumes.

According to one embodiment, the control system 140 is configured to reprogram the emission circuit 120 in such a way as to modify the emission aperture for each shot. This method then makes it possible to reduce the periodicity of the successive coherent sums and thus improve the quality of the reconstructed image by reducing for example the formation of grating lobes.

According to one embodiment, the control system 140 is configured to reprogram the reception circuit 130, and more specifically the reception and delay circuits of each group for each shot, in such a way as to modify the unfocused reception sub-apertures formed by groups of transducers or the elementary transducers chosen from each group for reception.

In the case of modifying reception sub-apertures, the control system 140 is configured to reprogram the electronic micro-beamforming circuits of each group for each shot in such a way as to modify the delays applied to the delay circuits 132C in order to modify the orientation of the convex reception surface and the radius of curvature thereof, as illustrated in the examples of FIGS. 6A-6C.

The control system 140 is configured to then coherently sum the 3D images obtained after each shot in order to generate a high-quality final image of the region.

The presence of the micro-beamforming circuits 131 in the probe 100 advantageously makes it possible to limit the number of signals to be transmitted to the control system 140 in a phase of receiving an ultrasound wave. The total number of signals to be transmitted, which corresponds to the number of transducers of the matrix, is divided by the number of groups of transducers.

All of the electrical signals generated by the micro-beamforming circuits 131 may be transmitted to the control system 140 in order to form the 3D image while at the same time limiting the size of the cable 5, making it possible to implement ultrafast imaging.

Moreover, the option of being able to reprogram the micro-beamforming circuits 131 by way of the control system 140 advantageously makes it possible to adjust the orientation of the virtual lenses and their radius of curvature as a function of the emission and reconstruction parameters of the desired 3D image.

FIGS. 7A and 7B illustrate another embodiment in which the control system 140 is configured to program the reception circuit 130 of FIG. 3 in such a way as to activate or address a reduced number of transducers of the array in order to supply a number of signals that corresponds to the number of channels available on the control system 140 of the device 1, thus making it possible to reduce the number of transmission channels between the probe 100 and the control system 140.

In this embodiment, the micro-beamforming circuits 131 located in the probe 100 are used to choose an elementary transducer to be activated at reception from among the elementary transducers in each group in order to reduce the number of response signals to be transmitted from the probe 100 to the control system 140. In other words, in the reception phase, the micro-beamforming reduction circuits 131 create a virtual reception array with a reduced number of signals to be transmitted from the probe 100 to the control system 140. The control system 140 therefore receives a set of signals coming from the virtual reception array.

The transducers are chosen from each group by the micro-beamforming circuits 131 according to an ordered addressing pattern or according to a random pattern as illustrated FIGS. 7A and 7B, respectively.

The micro-beamforming circuit 131 of FIG. 3 is programmed by the control system 140 in such a way as to address one transducer in each group. More specifically, the reception and delay circuits 132 are programmed in such a way as to individually address each elementary transducer of the group, that is to say to activate it or not activate it. By way of example, the time gain compensation circuit 132b of the reception and delay circuit 132 may be programmed so as to apply a zero gain to the response signal of the elementary transducer during the reception step so as not to activate an elementary transducer. The delay circuits 132c are programmed in such a way as not to apply delays to the elementary transducers.

According to one exemplary embodiment and as illustrated in FIG. 7A, the micro-beamforming circuit 131 of each group is programmed, by the control system, to address one transducer in each group in order to receive the waves backscattered by the medium. The elementary transducer activated to receive the backscattered waves is chosen according to an addressing pattern that is identical for all the groups of the array. The overall addressing pattern of the array is ordered. The total number of active transducers may be less than or equal to the number of available transmission channels. In the example illustrated, the white squares designate non-active transducers and the dark squares designate active transducers.

According to another exemplary embodiment and as illustrated in FIG. 7B, the micro-beamforming circuit 131 of each group is programmed, by the control system 140, to address one elementary transducer in each group in order to receive the waves backscattered by the medium. The transducer activated to receive backscattered waves is different from one group to another. The overall addressing pattern of the array is random.

According to one embodiment, the control system 140 is configured to reprogram the reception circuit 130, and more specifically the reception and delay circuits 132 of each group for each shot, in such a way as to modify the elementary transducers chosen from each group for reception.

According to one exemplary embodiment, the addressing pattern is identical for all groups in one and the same shot, but different between shots. In other words, for each group, the reception and delay circuits 132 are programmed to change the choice of transducer to be activated at each shot.

FIG. 7A illustrates a succession of the state of the elementary transducers corresponding to a succession of shots in the case of the example of an ordered addressing pattern. In a first shot, denoted T₁, the addressing pattern is repeated for all groups, namely the first elementary transducer 102₁₁ of each group is activated. In the following shot, denoted T₂, the addressing pattern (not visible in FIG. 7A) is different, and the micro-beamforming circuit is programmed in such a way as to activate for example the second elementary transducer 102₁₂. This addressing pattern is the same for all groups. In the last shot, denoted T₉, the micro-beamforming circuit is programmed in such a way as to activate for example the last elementary transducer of the group 102₃₃. This pattern is repeated for all other groups of this same shot.

FIG. 7B illustrates a succession of the state of the elementary transducers corresponding to a succession of shots in the case of the example of a random or pseudorandom addressing pattern. In the first shot, the addressing pattern is different for all groups, that is to say the activated elementary transducer is different for all groups in one and the same shot. For example, in the first group, it is the transducer denoted 102₃₂ that is activated whereas, in the second group, it is the transducer denoted 102₁₂ that is activated. In the next shot, denoted T₂, the addressing pattern (not visible in FIG. 7B) is different from that of the first shot.

Consequently, whether in the case of an ordered pattern or a random pattern, at each shot, the transducers that are activated at reception are different. As in the case of the embodiment illustrated in FIGS. 6A-6C, the final 3D image is then constructed from the coherent addition of a series of 3D images generated from the succession of shots so as to improve the quality of the 3D image.

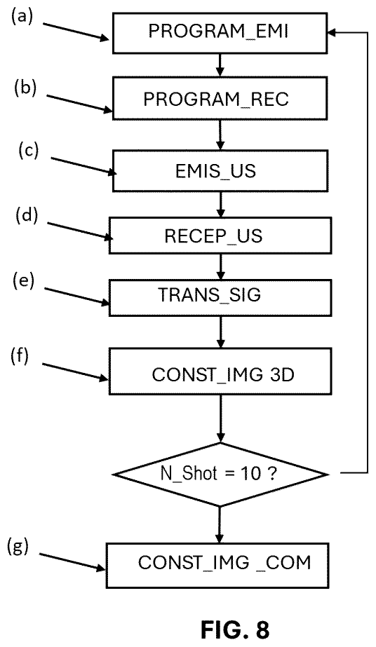

FIG. 8 illustrates the main steps of a volumetric ultrafast imaging method implementing the imaging device 1 of FIG. 2 and FIG. 3.

In an emission programming step (a) (PROGRAM_EMI), the control system 140 programs the emission circuit 120 to use a subset of adjacent elementary transducers or all the elementary transducers of the array 101 to transmit divergent ultrasound waves in the direction of the region to be imaged. The emission circuit 120 is programmed so as to activate the elementary transducers forming an emission aperture that determines the aperture angle of the ultrasound wave and designed to emit a plane or divergent unfocused ultrasound wave in the direction of the region to be imaged.

In the example of the array of FIG. 4A, the emission circuit 120 is programmed to activate the 9*9 elementary transducers so as to emit a plane or divergent ultrasound wave. In the example of FIG. 4B, the emission circuit 120 is programmed to activate some of the 9*9 elementary transducers so as to emit a plane or divergent ultrasound wave.

In a reception programming step (b) (PROGRAM_REC), the control system 140 programs the micro-beamforming circuits 131 associated with each group of transducers to apply a delay and summing operation such that each group of transducers emulates a virtual lens forming an unfocused reception sub-aperture or to choose an elementary transducer to be activated at reception from among the elementary transducers in each group according to an ordered or random pattern.

According to one embodiment, the control system 140 programs the micro-beamforming circuits 131 associated with each group of transducers to apply a delay and summation operation such that each group of transducers emulates a virtual acoustic lens having a reception surface of convex curved shape the focal point of which is located at the rear of the array, on the side opposite the medium to be imaged, as illustrated in FIGS. 6A-6C.

According to another embodiment, the control system 140 programs the micro-beamforming circuits 131 associated with each group of transducers in such a way as to select the elementary transducer of the group to be activated in order to obtain a desired distribution of active elementary transducers according to a predefined pattern, as illustrated by the examples of FIGS. 7A-7B.

In an ultrasound wave emission step (c) (EMIS_US), an unfocused ultrasound wave is emitted in the direction of the medium to be imaged by the array of activated elementary transducers by the emission circuit programmed by the control system. As a variant, the reception and emission programming steps (b) (PROGRAM_REC) and (c) (EMIS_US) may be carried out in the reverse order or simultaneously.

In a reception step (d) (RECEP_US), the backscattered signals are received by the transducers of each group, which forms an unfocused reception sub-aperture or a virtual lens. For each unfocused reception sub-aperture, the electrical signals that are generated may for example be amplified by the amplifier 132a and/or attenuated by the TGC circuit 132b. In the case of emulating a virtual lens, the delay circuits 132c then apply delays to the electrical signals. In the case of a distribution of elementary transducers activated according to a predefined pattern, the delay circuits 132c do not apply delays to the electrical signals. Finally, the delayed signals are summed by the summing circuit 133 so as to supply a single micro-formed analogue signal at the output of the micro-beamforming circuit 131.

In a transmission step (e) (TRANS_SIG), the signals supplied by all the groups are transmitted from the probe 100 to the control system 140 via the cable 5.

In a step of constructing a volume or a 3D image (f) (CONST_IMG_3D), the signals supplied by the groups of transducers are again delayed and summed in the control system 140 in order to construct a 3D image or a volume.

To improve the resolution and contrast of the image, steps (a) to (f) are repeated in order to make the transducer array successively emit a series of N unfocused ultrasound waves having different propagation directions and to make the transducer array receive a series of backscattered signals from said series of N successive unfocused ultrasound waves.

By way of example, each series may comprise for example 1 to 81 successive ultrasound waves of different directions, for example 3 to 25 successive ultrasound waves of different directions, for example 5 to 20 successive ultrasound waves of different directions, for example 10 to 20 successive ultrasound waves of different directions.

According to one embodiment, for each shot, the control system 140 reprograms the emission circuit to modify the emission aperture formed in the transducers of the array by modifying for example the size of the emission aperture and/or the emission delays applied to the elementary transducers.

According to one embodiment, for each shot, the control system 140 reprograms the emission circuit 120 to modify the emission delays applied to the elementary transducers in such a way as to modify the propagation direction of the ultrasound wave as indicated above. According to another embodiment, the elementary emission circuits are reprogrammed by the control system 140 such that the peripheral elementary transducers are not activated as in the case of FIG. 4B in such a way as to modify the dimension of the emission aperture.

According to one embodiment, for each shot, the control system 140 reprograms the reception circuit and more specifically the micro-beamforming circuits associated with each group of transducers to modify the reception sub-apertures and the reception delays to modify the orientation of the reception surface of the virtual lens formed by each group, as illustrated by the examples of FIGS. 6A-6C.

According to another embodiment, for each shot, the control system 140 reprograms the reception circuit and more specifically the micro-beamforming circuits associated with each group of transducers to modify the addressing pattern, as illustrated by the examples of FIGS. 7A and 7B.

In all cases, after each emission of ultrasound waves with a given propagation direction, backscattered echoes are acquired by the 2D array. These raw data (also called RF data or radiofrequency data) are first micro-formed in the probe 100, as indicated above in step (d), and then macro-formed again in the imaging system 140, as indicated in step (f), to generate a 3D image.

By way of example, after a series of 10 shots, that is to say 10 successive ultrasound waves of different propagation directions, 10 3D images are therefore formed.

In a step of constructing a high-quality final image (g) (CONST_IMG_COM), the method comprises a composite image construction step, during which 3D volumetric images are coherently composed so as to form a high-quality final 3D image.

Steps (a) to (f) are repeated at a high rate of between 5000 Hz and 20 000 Hz. The high rate is required in particular in ultrasound localization microscopy (ULM) imaging, and more generally to track the movement of tissue or fluids.

1. A device for three-dimensional ultrasound imaging of a region of interest of the body of a living being, comprising:
an ultrasound imaging probe comprising a plurality of elementary transducers arranged in a matrix in rows and columns forming an array, said elementary transducers being distributed into a set of groups of elementary transducers, an electronic emission circuit and an electronic reception circuit connected to the elementary transducers;
a control system configured to communicate with the ultrasound imaging probe;
wherein said electronic reception circuit comprises a micro-beamforming circuit for each group of elementary transducers, said micro-beamforming circuit having an input individually connected to each elementary transducer of the group and an output connected to the control system, said micro-beamforming circuit being designed to supply a single signal generated from the response signals of the elementary transducers of the group;
said control system being configured to program, for each shot:
(a) the electronic emission circuit to address a set of adjacent elementary transducers from among the plurality of elementary transducers in order to form an emission aperture and to apply a delay law to the elementary transducers of said set for the emission of an unfocused ultrasound wave;
(b) each electronic micro-beamforming circuit to address the associated group of elementary transducers or an elementary transducer of the group in order to form a reception sub-aperture having a focal point located behind the array, on a side opposite the region of interest to be imaged, designed to receive backscattered waves, such that a plurality of reception sub-apertures are formed.

2. The device according to claim 1, wherein the control system is configured to reprogram the electronic emission circuit to modify the emission aperture and the emission delays to emit an unfocused ultrasound wave at each shot.

3. The device according to claim 1, wherein the control system is configured to reprogram the electronic micro-beamforming circuits to modify the reception sub-apertures and the reception delays to receive backscattered waves at each shot.

4. The device according to claim 1, wherein the control system is configured to program the electronic emission circuit to apply emission delays to the transducers so that the emitted unfocused ultrasound wave is a plane wave or divergent wave.

5. The device according to claim 1, wherein each micro-beamforming circuit comprises a reception and delay circuit for each transducer of the group and a signal micro-summing circuit.

6. The device according to claim 5, wherein the reception and delay circuits of the group are capable of applying a delay law to the elementary transducers of the group to form a virtual lens the focal point of which is located behind the array, on the side opposite the region to be imaged.

7. The device according to claim 6, wherein the control system is configured to program the reception and delay circuits such that the virtual lenses that are formed have identical focal lengths.

8. The device according to claim 6, wherein the control system is configured to program the reception and delay circuits such that at least two virtual lenses have different focal lengths.

9. The device according to claim 6, wherein the control system is configured to program the reception and delay circuits such that at least two virtual lenses have different focusing directions.

10. The device according to claim 6, wherein the reception and delay circuit comprises a reception amplifier, a time gain compensation circuit and a delay circuit.

11. A method for three-dimensional (3D) ultrasound imaging of a region of interest of the body of a living being using a three-dimensional ultrasound imaging device according to claim 1, said method comprising, for each shot:
(a) a programming step during which the electronic emission circuit is programmed (PROGRAM_EMI) to address a set of adjacent elementary transducers from among the plurality of elementary transducers in order to form an emission aperture and to apply a suitable delay law to the elementary transducers of said set for the emission of an unfocused ultrasound wave;
(b) a programming step during which each electronic micro-beamforming circuit is programmed to address the associated group of elementary transducers or at least one elementary transducer of the group in order to form a reception sub-aperture having a focal point located behind the array, on a side opposite the region of interest to be imaged, designed to receive backscattered waves (PROGRAM_REC), such that a plurality of reception sub-apertures are formed;
(c) an emission step during which the unfocused ultrasound wave is emitted in the direction of the region of interest through said emission aperture formed by said set of addressed adjacent elementary transducers (EMI_US);
(d) a reception step during which the backscattered waves coming from the region of interest are received by each reception sub-aperture formed in the probe (REC_US);
(e) a signal transmission step during which the signal supplied by each micro-beamforming circuit is transmitted to the control system (TRANS_SIG);
(f) a 3D image construction step during which the signals are delayed and summed in the control system to construct a 3D ultrasound volumetric image corresponding to the emitted wave (CONST_IMG_COM).

12. The method according to claim 11, wherein steps (a) to (f) are repeated in order to emit a series of unfocused ultrasound waves in various propagation directions and to acquire a series of signals for each emitted wave, said signals making it possible to form a series of 3D volumetric images, and wherein the method furthermore comprises a step of constructing a composite image during which said 3D volumetric images are summed coherently in order to form a final composite image.

13. The method according to claim 12, wherein the control system reprograms the emission circuit to modify the emission aperture and the emission delays to emit an unfocused ultrasound wave at each shot.

14. The method according to claim 12, wherein the control system reprograms the electronic micro-beamforming circuit to modify the reception sub-apertures and the reception delays to receive backscattered waves at each shot.
